(19) United States
(12) Patent Application Publication
(10) Pub. No.: US 2025/0339043 A1
(43) Pub. Date: Nov. 6, 2025

(54) RANDOM-SEARCH ADAPTIVE TUNING FOR VOLUME CLAMP-BASED BLOOD PRESSURE MEASUREMENT

(71) Applicant: Becton, Dickinson and Company, Franklin Lakes, NJ (US)

(72) Inventors: Boris Reuderink, Amersfoort (NL); Hans Jean Paul Kuijkens, Amsterdam (NL); Jeroen Van Goudoever, Amsterdam (NL)

(21) Appl. No.: 19/266,805

(22) Filed: Jul. 11, 2025

Related U.S. Application Data
(63) Continuation of application No. PCT/US2024/011189, filed on Jan. 11, 2024.
(60) Provisional application No. 63/479,714, filed on Jan. 12, 2023.

(51) Int. Cl. A61B 5/0225; A61B 5/022; A61B 5/024

(52) U.S. Cl. CPC A61B 5/02255; A61B 5/02241; A61B 5/02422; A61B 2560/0223

(57) ABSTRACT

A control method includes encircling a patient appendage with a pressurizable cuff pressurized via a valve, and directing emitted light through a patient appendage to a light sensor. A sensed plethysmogram signal is generated based on the received light level, and the valve is controlled via a closed feedback loop responsive to an error value computed as the difference between the sensed plethysmogram signal and a calibrated setpoint value. An unperturbed error value characteristic is recorded through a first time window, and a random vector perturbation in the closed-loop control space is generated. A perturbed error value characteristic is recorded through a second time window while temporarily adjusting closed-loop control parameters based on the random vector perturbation. Baseline closed-loop control parameters are updated in response to the unperturbed error value characteristic exceeding the perturbed error value characteristic.

### CROSS-REFERENCE TO RELATED APPLICATION(S)

This application is a continuation of International Patent Application Number PCT/US2024/011189, filed Jan. 11, 2024, entitled “RANDOM-SEARCH ADAPTIVE TUNING FOR VOLUME CLAMP-BASED BLOOD PRESSURE MEASUREMENT,” which claims the benefit of U.S. Provisional Application No. 63/479,714, filed Jan. 12, 2023, and entitled “RANDOM-SEARCH ADAPTIVE TUNING FOR VOLUME CLAMP-BASED BLOOD PRESSURE MEASUREMENT,” the disclosures of which are hereby incorporated by reference in their entirety.

### BACKGROUND

The present disclosure relates generally to blood pressure sensing, and more particularly to an adaptive tuning method for volume clamp-based arterial blood pressure measurement.

Some non-invasive arterial blood pressure sensors generate a pressure reading by clamping (i.e. holding constant) arterial volume within a sensing region such as a portion of a finger surrounded by a pressurized cuff. Such systems indirectly assess arterial volume, e.g. optically, increasing or decreasing pressure applied by the pressurized cuff via closed-loop control to compensate for fluctuations in arterial volume caused by the pulsation of blood. The resulting clamping pressure allows blood pressure to be non-invasively monitored over long time periods, without interruption.

Arterial volume waveform is affected by numerous patient-specific factors, including arterial stiffness and blood flow. In addition, the shape of an individual patent's arterial volume waveform varies over time based on a multitude of factors including posture, cuff tightness, and physiological status of the patient. Control for clamp-based blood pressure measurement frequently involves selecting a model (herein referred to as a “plant model”) that accurately describes behavior of the sensed region (e.g., a portion of a patient finger) and the surrounding pressurized cuff. Model selection can be challenging, and errors in model selection produce unreliable sensor readings. Furthermore, model selection is generally only updated during open-loop recalibration that interrupts the closed-loop blood pressure sensing process described above. Conventional approaches hold control parameters constant during closed-loop volume clamping, and punctuate closed-loop clamping periods with open-loop calibration intervals to update these control parameters.

### SUMMARY

A control method for a hemodynamic monitoring system includes a light emitter, a light sensor, and a pressurizable cuff pressurized via a valve. The method includes encircling a patient appendage with a pressurizable cuff pressurized via a valve, and directing emitted light through a patient appendage to a light sensor. A sensed plethysmogram signal is generated based on the received light level, and the valve is controlled via a closed feedback control loop responsive to an error value computed as the difference between the sensed plethysmogram signal and a calibrated setpoint value. An unperturbed error value characteristic is recorded through a first time window, and a random vector perturbation in the control parameter space is generated. A perturbed error value characteristic is recorded through a second time window while temporarily adjusting PID control parameters based on the random vector perturbation. Baseline control parameters are updated in response to comparison of the perturbed and unperturbed error value characteristics.

A non-invasive sensor system includes a pressurizable cuff, a light emitter and a light sensor anchored to the pressurizable cuff, and a control module. The pressurizable cuff is pressurized via metered gas supply. The light sensor is positioned to receive light emitted by the light emitter and configured to generate a sensed plethysmogram signal based on received light. The control module controls actuation of the pressurizable cuff, and comprises a closed-loop controller, a window-based error assessor, and a closed-loop parameter adjuster. The closed-loop controller computes an error value as a difference between the sensed plethysmogram signal and a setpoint plethysmogram value, and actuates the metered gas supply based on closed-loop parameters and the error value. The window-based error assessor records a characteristic of the error value through multiple successive time windows. The closed-loop parameter adjuster provides the closed-loop controller with random temporarily adjustments to the closed-loop parameters, evaluate whether the characteristic of the error value is reduced by application of the random adjustment to the closed-loop parameters, and persistently update the closed-loop parameters according to the random adjustment to the closed-loop parameters when this evaluation indicating that the characteristic of the error value is reduced by application of the random adjustment to the closed-loop parameters.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a simplified perspective view of a non-invasive sensor system fitted to a human hand.

FIG. 2 is a schematic view of the non-invasive sensor system of FIG. 1 in operation.

FIG. 3 is a schematic view of a control system for the non-invasive sensor system of FIG. 2.

FIG. 4 is a process flowchart illustrating a method of operating the control system of FIG. 3.

FIGS. 5A-D are graphs illustrating example parameters and results of adaptive control according to the method of FIG. 4.

While the above-identified figures set forth one or more examples of the present disclosure, other examples are also contemplated, as noted in the discussion. In all cases, this disclosure presents methods and apparatus by way of representation and not limitation. It should be understood that numerous other modifications, structures, and methods can be devised by those skilled in the art, which fall within the scope and spirit of the principles of the present disclosure. The figures may not be drawn to scale, and applications and examples of the present disclosure may include features and components not specifically shown in the drawings.

### DETAILED DESCRIPTION

The present disclosure describes an approach to volume clamping for a non-invasive arterial blood pressure sensing system. This approach eschews plant model selection in favor of generating and testing random perturbations to closed-loop controller parameters. In the examples provided below, volume clamping is accomplished using closed-loop control. The present approach generates random perturbations to control parameters, evaluates sensing error, and updates baseline closed-loop control with perturbations that lower sensing error. This approach avoids reliance on a priori knowledge of the overall system (plant), and permits control adaptation without interrupting closed-loop control.

Existing systems use methods to gain knowledge about the plant by introducing specific conditions such as variations in open loop control or perturbations in control signal. By contrast, the approach set forth below adjusts a baseline closed-loop control by assessing the consequences of small random perturbations in the control coefficients of the closed-loop controller, rejecting perturbations that increase error and incorporating perturbations that reduce error into baseline closed-loop control.

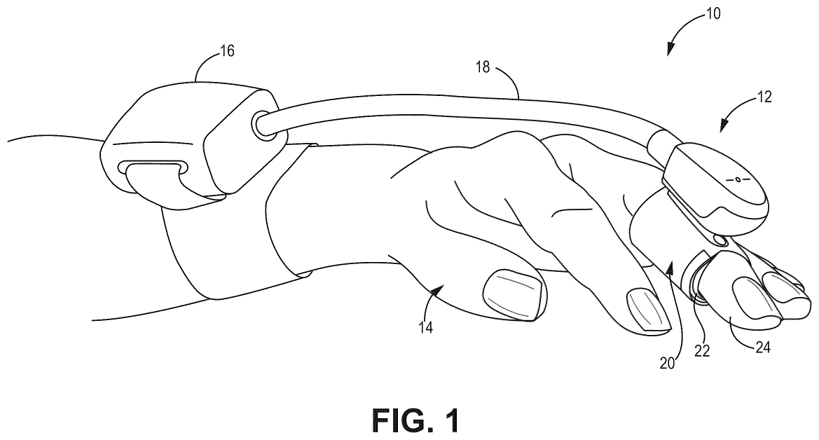

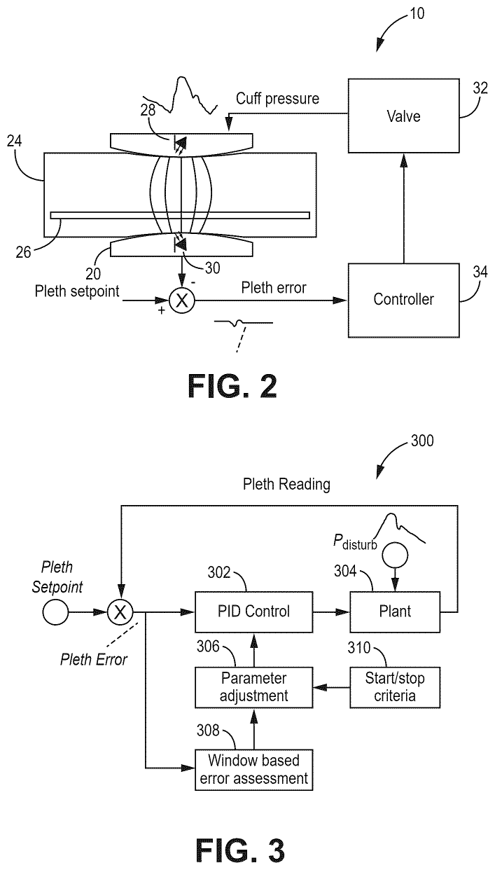

FIG. 1 provides a simplified perspective view sensor system 12 attached to hand 14. FIG. 2 is a schematic view of sensor system 12 in operation. FIGS. 1 and 2 are primarily described together. As shown in FIG. 1, sensor system 12 is a non-invasive hemodynamic sensor capable of generating arterial blood pressure measurements through volume clamping. Sensor system 12 can include housing 16, connector 18, cuff 20, and pressurizable bladder 22. In the illustrated example, cuff 20 is a ring or similar structure surrounding or bracketing finger 24 of hand 14, while housing 16 is a wrist-mounted device coupled to cuff 20 via connector 18. In the most general case, however, sensor system 12 can differ substantially from the layout illustrated in FIG. 1. Sensor system 12 can, for example, include multiple separate connectors 18 between elements attached to finger 24 (e.g. cuff 20), and/or can relocate housing 16 to other locations (e.g. integrated with cuff 20, or separately disposed at a peripheral location). In the illustrated example, cuff 20 surrounds a sensing region of a finger 24 of hand 14. At least one artery 26 passes through the sensing region. Cuff 20 also anchors pressurizable bladder 22, which can for example be an expandable annular air bladder fed by an air line included within connector 18, or from another source. In the most general case, however, pressurizable bladder 22 can be any sort of mechanism suited to apply pressure to finger 24 based on control as described below. Sensor system 12 and hand 14 together make up combined physical system 10 (sometimes referred to as a plant or plant system) responsive both to changes in the patient and change in control of sensor system 12.

As shown in FIG. 2, cuff 20 includes light emitter 28 and light sensor 30. Light emitter 28 emits light through the sensing region (denoted in FIG. 2 by path lines through finger 24) for reception by light sensor 30. The wavelengths of light emitted by light emitter 28 can all fall within the visible-to-infrared spectrum. Light sensor 30 detects received amplitude of light from light emitter 28. In some examples, light emitter 28 and light sensor 30 can be situated on opposite sides of cuff 20, such that light travels directly through the sensing region of finger 24 from light emitter 28 to light sensor 30. More generally, however, scattering of light from light emitter 28 inside tissue of finger 24 allows light emitter 28 and light sensor 30 to be effective even when not disposed on opposite sides of cuff 20, e.g. when located proximate one another.

Overall light amplitude received at light sensor 30 from transmission by emitter 28 is referred to hereinafter as pleth (plethysmogram) signal, and is used as a proxy for inverse arterial volume within the sensing region, with a reduction in received light corresponding to an increase in arterial volume. The two arteries of the finger and connected capillaries pulsate during normal blood flow, expanding (with systolic pressure) and relaxing (with diastolic pressure) in volume over the course of each heartbeat cycle. Greater arterial volume increases absorption of emitted light, reducing the fraction of emitted light received at light sensor 30.

As principally described herein, sensor system 12 clamps arterial volume within the sensing region by inflating pressurizable bladder 22 through actuation of valve 32. In some examples, actuation of valve 32 modulates air pressure provided through a pneumatic line contained within connector 18. Valve 32 can, for example, be a servo valve. In the most general case, however, valve 32 can be any sort of metering device capable of controlling pressurization of cuff 22. Valve 32 can, in an alternative example, be a piezo pump.

Sensor system 12 includes controller 34, with logic-capable hardware configured to adjust settings of valve 32 in a control loop responsive to the pleth reading. Controller 34 can, for example, be a control module instantiated in dedicated hardware, or a software module running on hardware within sensor system 12. In some examples valve 32 and controller 34 can both be situated within housing 16. More generally, however, valve 32 can be located in any suitable location to meter pressurization of pressurizable bladder 22, and controller 34 can be located within housing 16, within or closer to cuff 22, or at any other location capable of supporting processing to control actuation of valve 32. In some examples, some functions of controller 34 can offloaded to a peripheral device (not shown in FIG. 1).

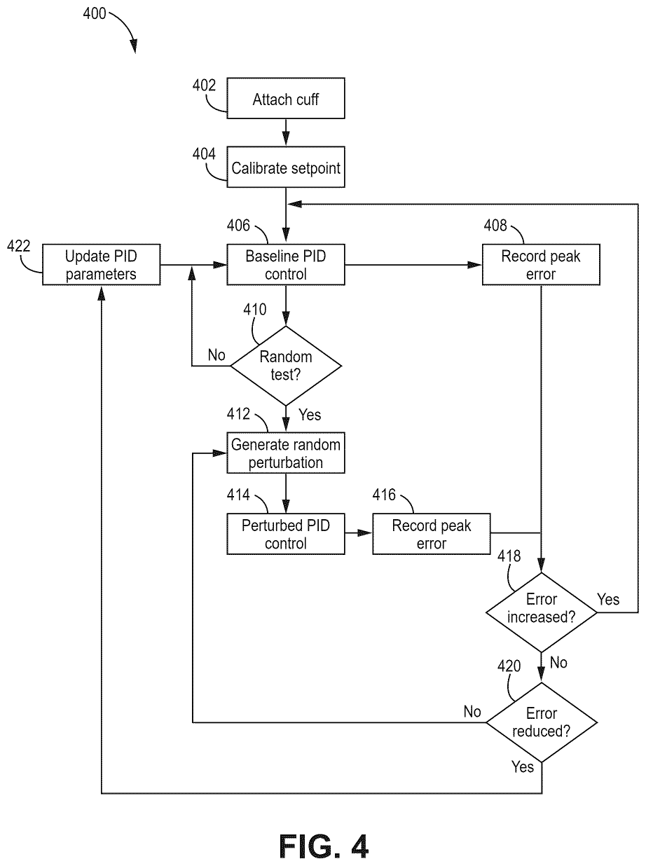

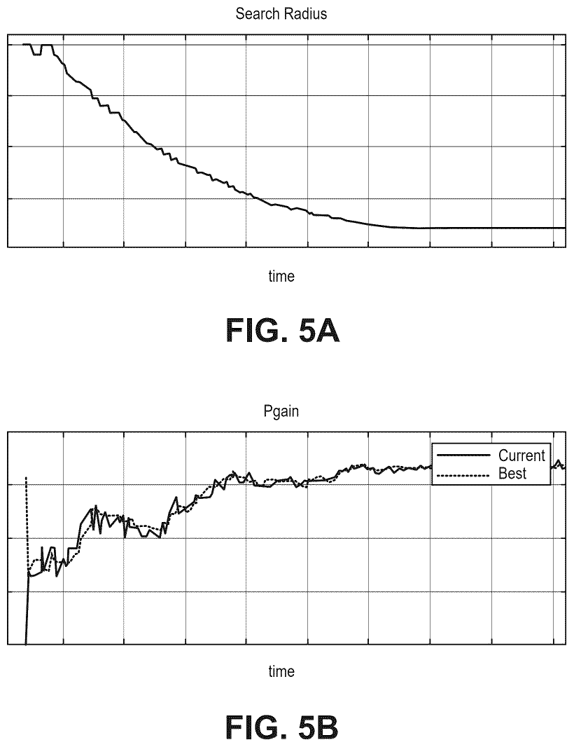

Increased flow through valve 32 into pressurizable bladder 22 results in expansion of pressurizable bladder 22, physically constricting finger 24 and thereby arterial volume in the sensing region. A difference (identified hereinafter as “pleth error”) between the pleth signal and a corresponding target setpoint is used as an input for control of valve 32 by controller 34. In the detailed description provided hereinafter this control scheme and method (see FIGS. 3 and 4) are described as involving proportional-integral-derivative (PID) control based on the pleth error. In the most general case, however, other forms of closed-loop control can be substituted for PID control. Controller 34 sets pressure applied by pressurizable bladder 22 through actuation of valve 32 to mechanically oppose changes in arterial volume within the sensing region, reducing the magnitude of arterial volume fluctuation. The valve pressure generated by this clamping process serves as a measure of arterial blood pressure. As explained in detail below, the present disclosure provides a method and system for adaptively tuning parameters of this closed-loop control loop via an iterative random-search.

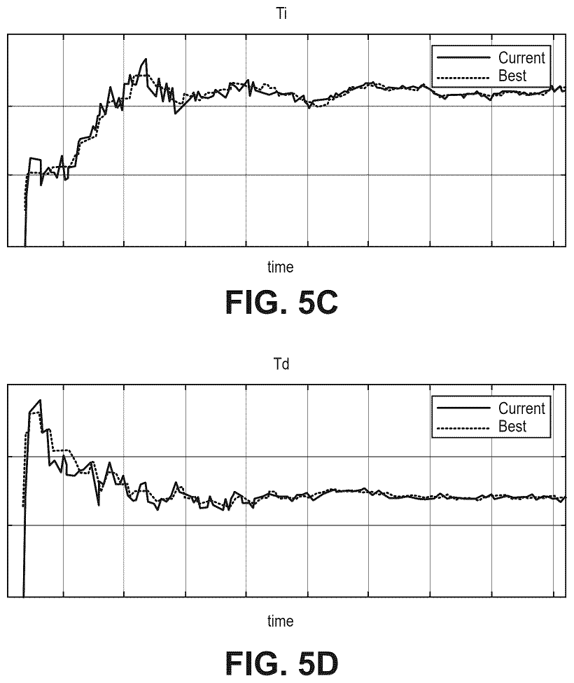

FIG. 3 is functional block diagram illustrating control process 300 for non-invasive sensor system 10. FIG. 4 is a flowchart illustrating method 400, an example method of operation for non-invasive sensor system 10 using control process 300. FIGS. 5A-D are graphs illustrating example operating parameters and characteristics of adaptive PID control according to method 400, as a function of time. More specifically, FIG. 5A illustrates search random perturbation search radius and FIGS. 5B-D illustrate P, I, and D terms of PID control 302, respectively. FIGS. 3, 4, and 5A-D are primarily described together.

FIG. 3 depicts closed-loop system 300 whereby PID control 302 controls plant 304, resulting in changes in the pleth signal that generate a pleth error as described above when compared to a pleth setpoint. This pleth error feeds back into PID control 302 for subsequent control. Plant 304 describes the physical combination of hand 14 and sensor system 12, particularly including pressurizable bladder 22. PID control 302, which can be instantiated in software or hardware of controller 34 (see FIG. 2), is referred to and described herein primarily as a proportional-integral-derivative controller. As noted above, however, the approaches set forth herein can also be applied to similar systems such as proportional-integral (PI), proportional-derivative (PD), and proportional-integral-feed-forward (PIFF) control schemes. In the most general case, the present disclosure is applicable to closed- or partially closed-loop control systems having a defined space of control parameters. Referring to the example of proportional-integral-derivative control, specifically, PID control 302 drives behavior of plant 304 (through actuation of valve 32 and thereby pressurizable bladder 22) by production of a valve gain signal dependent both on the current pleth error and on proportional, integral, and derivative control parameters. These parameters—that is, coefficients of the control algorithm run by PID control 302—are adaptively tuned by parameter adjustment module 306 based on error analysis provided by window-based error assessment module 308. Parameter adjustment module 306 need not be in continuous operation. As described in greater detail below, parameter adjustment module 306 can stop or start adaptive tuning of PID control parameters based on start/stop criteria 310. In this way, system 300 only engages in adaptive searching for improved PID control parameters under certain circumstances, e.g., if error assessment indicates that control is inadequate, and subsequently halts once PID control is satisfactory.

Closed-loop system 300 operates according to control method 400, which is illustrated by FIG. 4. Control method 400 begins with the attachment of pressurizable bladder 22 about finger 24 of hand 14 (Step 402), and proceeds with calibration to determine the pleth setpoint (Step 404). This calibration process can, for example, be an open-loop process wherein the pleth signal is monitored through multiple patient heartbeats while pressurization or constriction of pressurizable bladder 22 is held constant or varied according to a known pattern or sequence not set by the closed-loop control algorithm. This open-loop calibration process is used both for initial setup, i.e. to ascertain an initial setpoint for a new patient, and periodically during the monitoring of a patient to adjust for changes in patient condition. The pleth setpoint ideally corresponds to a relaxed arterial volume (i.e. undilated by arterial pulsations and not compressed by the surrounding cuff) within the sensing region for present conditions of finger 24, e.g. including patient hand position/posture and blood perfusion. Closed-loop volume clamping can also be interrupted for open-loop calibration both on a scheduled basis to adjust for small changes in patient position or condition, and on a triggered (non-scheduled) basis in response to irregularities indicating that the current pleth setpoint requires recalibration.

Once a pleth setpoint has been set, method 400 is able to proceed to baseline closed-loop volume clamping control (Step 406). In this mode, PID control 302 adjusts plant behavior by controlling gain of valve 32, thereby causing changes to the pleth reading reflected in a pleth error that feeds back into PID control 302. More specifically, PID control 302 drives constriction of pressurizable bladder 22 to counteract systolic dilation of arterial volume within the sensing region of finger 24 as indicated by a reduction in light level detected by light sensor 30, and relaxes (i.e., reduces pressurization of) pressurizable bladder 22 during diastolic contraction of the arterial volume. In this way, PID control 302 facilitates clamping arterial volume, preventing or reducing physical pulsation of the artery in the sensing region. The resulting pressure applied by cuff 22 to counteract arterial volume expansion corresponds to arterial blood pressure.

As noted above, parameter adjustment module 306 adjusts control parameters by generating a random perturbation to PID (or analogous) control parameters and testing the effect of this perturbation based on sensed pleth values, using at least one cost function. Perturbations resulting in a worsening of performance according to the selected cost function(s) are rejected, while perturbations resulting in an improvement in performance are accepted as a new baseline for the PID control parameters. Cost functions are evaluated over a sensing time window, and can include, but are not limited to: (1) mean square or absolute pleth error; (2) maximum peak-to-peak pleth error amplitude; (3) percentage of time with pleth error exceeding a preset threshold; (4) peak positive or negative pleth error; (5) derived parameters from pleth waveform, e.g. maximal rate of change of pressure as a function of time; and (6) detections or counts of pleth signal oscillations attributable to gain-based overshoot. Combinations of such cost functions can also be used, for example as contributors to an aggregate cost function, as alternatives evaluated contingently or situationally, and/or as parallel cost functions capable of independently triggering rejection of a control value perturbation. For simplicity of explanation, subsequent discussion assumes that peak pleth error amplitude within the selected time window is the sole cost function evaluated by parameter adjustment module 306. Method 400 can be generalized, however, to any cost function or combination of cost functions as set forth above.

Parameter adjustment module 306 adjusts control parameters (e.g., P, I, and D coefficients) of PID control 302 during the aforementioned closed-loop volume clamping mode of operation of sensor system 12. In some examples, parameter adjustment module 306 can also adjust control parameters during the open-loop calibration mode. Initial conditions for PID control parameters can be determined from a variety of data, including both a priori knowledge regarding sensor system 12 and/or hand 14 (or the patient, more generally), and data obtained from initial open-loop calibration, including but not limited to cuff pressure/tightness, sensed arterial volume (or sensed pleth values, as proxies therefor), and physiological status of the finger more generally (e.g. perfusion, orientation, vein stiffness). Window-based error assessment module 308 records a cost parameter based on the pleth signal during baseline PID control. (Step 408). Carrying forward the example set forth above, this measure can for example be a peak pleth error amplitude within a sensing period.

In an illustrative example, window-based error assessment module 308 receives sensed pleth values during a baseline closed-loop (e.g. PID) volume clamping control of sensor system 12 through a baseline sensing time window, and records peak pleth error amplitude during that time window. (Step 408). Parameter adjustment module 306 evaluates whether parameter adjustment may be appropriate based on start/stop criteria 310. (Step 410). Start/stop criteria 310 include triggers for engaging adaptive tuning (i.e., start criteria) and triggers for halting adaptive tuning (i.e. stop criteria). Start and stop criteria can be aggregative, i.e. requiring that all or multiple criteria are satisfied to trigger a start or stop of operation for parameter adjustment module 306. Alternatively or additionally, specific start or stop criteria can serve as independently sufficient triggers to engage or halt parameter adjustment, respectively.

Start criteria can, for example, include initialization of sensor system 12, e.g., after attaching cuff 22 (step 402) to a patient. Start criteria can also include indications based on pleth error or sensed pleth signal waveform that plant dynamics have changed significantly, e.g., triggered by a change in position of hand 14 or a change in vasoconstriction caused by medication. A spike in pleth error over a threshold value, sustained over multiple heartbeats, can serve as such an indicator. Start criteria can incorporate comparisons of closed-loop and open-loop pleth signals. In an illustrative example, a ratio of closed- to open-loop peak-to-peak pleth error values rising above a “start” threshold can be a start criterion.

Stop criteria are selected to avoid instability or noise, and are used to disable parameter adjustment module 306 when further adaptation to optimize PID parameters is unnecessary or unlikely to be successful. In an illustrative example, a ratio of closed- to open-loop peak-to-peak pleth error values falling below above a “stop” threshold (lower than the “start” threshold noted above) can be a stop criterion. In another example, a stop criterion can include rate of change (or change per time interval) of a ratio of PID parameter vector magnitude to resulting peak-to-peak error falling below a threshold value, indicating that parameter adaptation effectiveness has fallen to below an acceptable level.

If the stop criteria indicate that adaptive tuning should be halted, or if adaptive tuning has not yet begun based on selected start criteria, no random test is initiated and baseline PID control continues unabated (i.e., step 410 proceeds back to step 406). Conversely, if start/stop criteria indicate that adaptive tuning is appropriate, parameter adjustment module 306 initiates a random test by generating a random perturbation to closed-loop control parameters. (Step 412). Random perturbations take the form of vectors in the control parameter space, applied to the current baseline control parameters (i.e., as operating in step 406). Random PID perturbations, for example, consist of random factors and/or additive constants applied to proportional, integral, and derivative control coefficients. These random vector perturbations within the control parameter space can be generated using a variety of approaches, including genetic algorithms, simulated annealing, Luus-Jaakola optimization, and particle swarm optimization. The resulting vector perturbation is applied as a temporary correction to PID control parameters, resulting in a perturbed closed-loop PID control mode. (Step 414). While PID control 302 operates in this perturbed state, window-based error assessment module 308 records the cost parameter previously recorded during baseline PID control, at step 408. This cost parameter can for example be a peak pleth error amplitude within a sensing period. The sensing periods to generate the recorded cost parameters in steps 408 and 416 preferably include at least two patient heartbeats. In some examples, these sensing periods can include eight or more patient heartbeats to minimize variation due to respiration.

Parameter adjustment module 306 compares cost values associated with the perturbed and unperturbed PID control periods. Carrying forward the primary example set forth herein, parameter adjustment module 306 first evaluates whether the control parameter vector perturbation resulted in a reduction in an increase in peak pleth error during the sampled time windows. (Step 418). If so, method 400 reverts to stable PID parameters for baseline PID control 406 to avoid instabilities. Next, parameter adjustment module 306 evaluates whether the control parameter vector perturbation resulted in a reduction in peak pleth error during the sampled time windows. (Step 420). If not, the perturbation is discarded and a new perturbation is generated. Although FIG. 4 illustrates the generation of a new random perturbation proceeding immediately from the discarding of an unproductive perturbation, control process 300 can in some examples or situations first return to baseline PID control (step 406) to reestablish corresponding peak pleth error (408) before proceeding to test a new random perturbation. According to some random-search algorithms, discarded perturbations can influence the selection of perturbations generated for testing in future adaptive tuning iterations.

If comparison indicates that the control parameter vector perturbation resulted in a reduction in peak pleth error across the sampled time windows, the baseline PID control parameters are updated (step 422) before any subsequent perturbations are tested—that is, the perturbed PID control parameters are retained as new baseline PID control parameters.

In some examples, depending on the random-search algorithm selected, the magnitude of tested perturbation vectors can be reduced over time as shown in FIG. 5A. In this fashion the step size of subsequent iterative PID control parameter improvements is gradually reduced to avoid overshoot and allow control parameters to efficiently converge upon a satisfactory value. This convergence can be seen in FIGS. 5B-D, which illustrate example values of proportional, integral, and derivative PID coefficients adjusted over many steps by comparison of current (solid) to historical best (dotted) values of these parameters, by cost.

The systems and methods set forth herein facilitate adaptive improvement to control parameters without reliance on correct identification or estimation of a plant model, and without reliance on interruption of closed-loop volume clamping.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### Discussion of Detailed Examples

The following are non-exclusive descriptions of possible examples of the present disclosure.

A method of controlling a hemodynamic monitoring system including a light emitter, a light sensor, and a pressurizable cuff pressurized via a metering device, the method comprising: encircling a patient appendage with the pressurizable cuff; directing light emitted by the light emitter through the patient appendage; generating a sensed plethysmogram signal based on light level received by the light sensor through the patient appendage; calibrating a setpoint plethysmogram value; controlling the metering device, and thereby pressurization of the pressurizable cuff, via a feedback loop including a closed loop controller responsive to an error value computed as a difference between the sensed plethysmogram signal and the setpoint plethysmogram value; recording an unperturbed error value characteristic through a first time window; generating a random vector perturbation within a parameter space of control parameter terms of the closed-loop controller; temporarily adjusting control parameters of the closed-loop controller according to the random vector perturbation, thereby adjusting arterial volume clamping provided by the pressurizable cuff, and recording a perturbed error value characteristic, through a second time window; in response to the unperturbed error value characteristic exceeding the perturbed error value characteristic, updating baseline values of the control parameters of the closed-loop controller according to the random vector perturbation.

The method of the preceding paragraph can optionally include, additionally and/or alternatively, any one or more of the following features, configurations and/or additional components:

A method as set forth above, wherein the unperturbed error value characteristic and the perturbed error value characteristic comprise peak error values within the first and second time windows, respectively.

A method as set forth above, wherein the sensed plethysmogram signal is a proxy for arterial volume within the appendage, based on sensed light level, and the setpoint plethysmogram signal corresponds to an arterial rest volume.

A method as set forth above, wherein calibrating the setpoint plethysmogram value comprises determining the arterial rest volume during an open loop calibration period in which the light emitter and the light sensor are active and closed-loop control of the metering device is disabled.

A method as set forth above, wherein the first and second test windows each have duration longer than a period of at least two heartbeats of the patient.

A method as set forth above, further comprising iteratively repeating, in sequence, the steps of recording the unperturbed error, generating a random vector perturbation, temporarily adjusting the control parameters according to the random vector perturbation, and updating the baseline values of the control parameters.

A method as set forth above, wherein the random vector perturbation is generated at each iteration according to a random walk approach selected from the group consisting of genetic algorithms, simulated annealing, Luus-Jaakola optimization, and particle swarm optimization.

A method as set forth above, wherein a maximum vector magnitude of the random vector perturbation is reduced with each successive iteration, after updating baseline values of the control parameters of the closed-loop controller according to the random vector perturbation.

A method as set forth above, wherein the iterative repeating of steps is halted when the maximum vector magnitude of the random vector perturbation is reduced below a minimum value.

A method as set forth above, further comprising monitoring an open-loop peak-to-peak error value while calibrating the setpoint plethysmogram value, and a closed-loop peak-to-peak error value during operation of the closed-loop controller, wherein iterative repeating of steps is halted in response to a ratio of the open-loop peak-to-peak error value to the closed-loop peak-to-peak error value falling below a preset threshold ratio.

A method as set forth above, further comprising monitoring an open-loop peak-to-peak error value while calibrating the setpoint plethysmogram value, and a closed-loop peak-to-peak error value during operation of the closed-loop controller, wherein the generating of a random vector perturbation and the temporary adjustment of control parameters of the closed-loop controller according to the random vector perturbation are triggered in response to a ratio of the open-loop peak-to-peak error value to the closed-loop peak-to-peak error value exceeding a preset threshold ratio.

A method as set forth above, wherein the generating of a random vector perturbation and the temporary adjustment of control parameters of the closed-loop controller according to the random vector perturbation are also triggered at an initial calibration of the hemodynamic monitoring system, after encircling the patient appendage with the pressurizable cuff.

A method as set forth above, wherein the closed-loop controller is a proportional-integral-derivative (PID) controller, and wherein the control parameter terms of the closed-loop controller are proportional, integral, and/or derivative terms of the PID controller.

A method as set forth above, further comprising, in response to the perturbed error value characteristic exceeding the unperturbed error value characteristic, reverting to closed-loop control using baseline, unperturbed control parameters of the closed-loop controller.

A non-invasive sensor system comprising: a pressurizable cuff pressurized via a metered gas supply; a light emitter anchored to the pressurizable cuff; a light sensor anchored to the pressurizable cuff, positioned to receive light emitted by the light emitter, and configured to generate a sensed plethysmogram signal therefrom; and a control module configured to control actuation of the pressurizable cuff to clamp arterial volume surrounded by the cuff, the control module comprising: a PID controller configured to compute an error value as a difference between the sensed plethysmogram signal and a setpoint plethysmogram value, and to actuate the metered gas supply based on PID parameters and the error value; a window-based error assessor configured to record a characteristic of the error value through multiple successive time windows; and a PID parameter adjuster configured to temporarily provide the PID controller with a random adjustment to the PID parameters, evaluate whether the characteristic of the error value is reduced by application of the random adjustment to the PID parameters, and persistently update the PID parameters according to the random adjustment to the PID parameters in response to the evaluation indicating that the characteristic of the error value is reduced by application of the random adjustment to the PID parameters.

The non-invasive sensor system of the preceding paragraph can optionally include, additionally and/or alternatively, any one or more of the following features, configurations and/or additional components:

A non-invasive sensor system as set forth above, wherein the pressurizable cuff is sized to fit a patient appendage, such that the light emitter is directed through the patient appendage toward the light sensor.

A non-invasive sensor system as set forth above, wherein the characteristic of the error value is a peak error value within each successive time window, and evaluation of whether the characteristic of the error value is reduced by application of the random adjustments comprises comparison of a peak error recorded by the window-based error assessor during a time window in which the random adjustment is applied to the PID parameters, to a peak error recorded by the window-based error assessor during a time window in which the random adjustment is not applied to the PID parameters.

A non-invasive sensor system as set forth above, wherein each of the successive time windows has a duration longer than two patient heartbeats.

A non-invasive sensor system as set forth above, further comprising a stop/start trigger configured to evaluate peak-to-peak error and disable and/or enable the PID parameter adjuster based on the evaluation of peak-to-peak error.

A non-invasive sensor system as set forth above, wherein the evaluation of peak-to-peak error comprises a comparison between a closed-loop (PID) peak-to-peak error value and an open-loop peak-to-peak error value.

A non-invasive sensor system as set forth above, wherein the PID parameter adjuster generates the random adjustment according to an approach selected from the group consisting of genetic algorithms, simulated annealing, Luus-Jaakola optimization, and particle swarm optimization.

A non-invasive sensor system as set forth above, wherein the PID parameter adjuster generates the random adjustment to the PID parameters as a vector within the PID parameter space, the vector having a maximum magnitude that decreases across multiple iterations of successive random adjustments.

While the invention has been described with reference to exemplary examples, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular examples disclosed, but that the invention will include all approaches falling within the scope of the appended claims.

1. A method of controlling a hemodynamic monitoring system including a light emitter, a light sensor, and a pressurizable cuff pressurized via a metering device, the method comprising:
encircling a patient appendage with the pressurizable cuff;
directing light emitted by the light emitter through the patient appendage;
generating a sensed plethysmogram signal based on light level received by the light sensor through the patient appendage;
calibrating a setpoint plethysmogram value;
controlling the metering device, and thereby pressurization of the pressurizable cuff, via a feedback loop including a closed loop controller responsive to an error value computed as a difference between the sensed plethysmogram signal and the setpoint plethysmogram value;
recording an unperturbed error value characteristic through a first time window;
generating a random vector perturbation within a parameter space of control parameter terms of the closed-loop controller;
temporarily adjusting control parameters of the closed-loop controller according to the random vector perturbation, thereby adjusting arterial volume clamping provided by the pressurizable cuff, and recording a perturbed error value characteristic, through a second time window; and
in response to the unperturbed error value characteristic exceeding the perturbed error value characteristic, updating baseline values of the control parameters of the closed-loop controller according to the random vector perturbation.

2. The method of claim 1, wherein the unperturbed error value characteristic and the perturbed error value characteristic comprise peak error values within the first and second time windows, respectively.

3. The method of claim 1, wherein the sensed plethysmogram signal is a proxy for arterial volume within the appendage, based on sensed light level, and the setpoint plethysmogram signal corresponds to an arterial rest volume.

4. The method of claim 3, wherein calibrating the setpoint plethysmogram value comprises determining the arterial rest volume during an open loop calibration period in which the light emitter and the light sensor are active and closed-loop control of the metering device is disabled.

5. The method of claim 1, wherein the first and second test windows each have duration longer than a period of at least two heartbeats of the patient.

6. The method of claim 1, further comprising iteratively repeating, in sequence, the steps of recording the unperturbed error, generating a random vector perturbation, temporarily adjusting the control parameters according to the random vector perturbation, and updating the baseline values of the control parameters.

7. The method of claim 6, wherein the random vector perturbation is generated at each iteration according to a random walk approach selected from the group consisting of genetic algorithms, simulated annealing, Luus-Jaakola optimization, and particle swarm optimization.

8. The method of claim 6, wherein a maximum vector magnitude of the random vector perturbation is reduced with each successive iteration, after updating baseline values of the control parameters of the closed-loop controller according to the random vector perturbation.

9. The method of claim 6, wherein the iterative repeating of steps is halted when the maximum vector magnitude of the random vector perturbation is reduced below a minimum value.

10. The method of claim 6, further comprising monitoring an open-loop peak-to-peak error value while calibrating the setpoint plethysmogram value, and a closed-loop peak-to-peak error value during operation of the closed-loop controller, wherein iterative repeating of steps is halted in response to a ratio of the open-loop peak-to-peak error value to the closed-loop peak-to-peak error value falling below a preset threshold ratio.

11. The method of claim 6, further comprising monitoring an open-loop peak-to-peak error value while calibrating the setpoint plethysmogram value, and a closed-loop peak-to-peak error value during operation of the closed-loop controller, wherein the generating of a random vector perturbation and the temporary adjustment of control parameters of the closed-loop controller according to the random vector perturbation are triggered in response to a ratio of the open-loop peak-to-peak error value to the closed-loop peak-to-peak error value exceeding a preset threshold ratio, wherein the generating of a random vector perturbation and the temporary adjustment of control parameters of the closed-loop controller according to the random vector perturbation are also triggered at an initial calibration of the hemodynamic monitoring system, after encircling the patient appendage with the pressurizable cuff.

12. The method of claim 1, wherein the closed-loop controller is a proportional-integral-derivative (PID) controller, and wherein the control parameter terms of the closed-loop controller are proportional, integral, and/or derivative terms of the PID controller.

13. The method of claim 1, further comprising, in response to the perturbed error value characteristic exceeding the unperturbed error value characteristic, reverting to closed-loop control using baseline, unperturbed control parameters of the closed-loop controller.

14. A non-invasive sensor system comprising:
a pressurizable cuff pressurized via a metered gas supply;
a light emitter anchored to the pressurizable cuff;
a light sensor anchored to the pressurizable cuff, positioned to receive light emitted by the light emitter, and configured to generate a sensed plethysmogram signal therefrom; and
a control module configured to control actuation of the pressurizable cuff to clamp arterial volume surrounded by the cuff, the control module comprising:
a PID controller configured to compute an error value as a difference between the sensed plethysmogram signal and a setpoint plethysmogram value, and to actuate the metered gas supply based on PID parameters and the error value;
a window-based error assessor configured to record a characteristic of the error value through multiple successive time windows; and
a PID parameter adjuster configured to temporarily provide the PID controller with a random adjustment to the PID parameters, evaluate whether the characteristic of the error value is reduced by application of the random adjustment to the PID parameters, and persistently update the PID parameters according to the random adjustment to the PID parameters in response to the evaluation indicating that the characteristic of the error value is reduced by application of the random adjustment to the PID parameters.

15. The non-invasive sensor system of claim 14, wherein the pressurizable cuff is sized to fit a patient appendage, such that the light emitter is directed through the patient appendage toward the light sensor.

16. The non-invasive sensor system of claim 14, wherein the characteristic of the error value is a peak error value within each successive time window, and evaluation of whether the characteristic of the error value is reduced by application of the random adjustments comprises comparison of a peak error recorded by the window-based error assessor during a time window in which the random adjustment is applied to the PID parameters, to a peak error recorded by the window-based error assessor during a time window in which the random adjustment is not applied to the PID parameters.

17. The non-invasive sensor system of claim 14, further comprising a stop/start trigger configured to evaluate peak-to-peak error and disable and/or enable the PID parameter adjuster based on the evaluation of peak-to-peak error.

18. The non-invasive sensor system of claim 17, wherein the evaluation of peak-to-peak error comprises a comparison between a closed-loop (PID) peak-to-peak error value and an open-loop peak-to-peak error value.

19. The non-invasive sensor system of claim 14, wherein the PID parameter adjuster generates the random adjustment according to an approach selected from the group consisting of genetic algorithms, simulated annealing, Luus-Jaakola optimization, and particle swarm optimization.

20. The non-invasive sensor system of claim 14, wherein the PID parameter adjuster generates the random adjustment to the PID parameters as a vector within the PID parameter space, the vector having a maximum magnitude that decreases across multiple iterations of successive random adjustments.
